(19) United States
(12) Patent Application Publication
(10) Pub. No.: US 2015/0051537 A1
(43) Pub. Date: Feb. 19, 2015

(54) CATHETER FLOW CONTROLLING

(71) Applicant: COVIDIEN LP, Mansfield, MA (US)

(72) Inventor: Brent Marsden, Reading, MA (US)

(21) Appl. No.: 13/969,779

(22) Filed: Aug. 19, 2013

(51) Int. Cl. A61M 1/36; A61M 39/28; A61M 25/00

(52) U.S. Cl. CPC A61M 1/3661; A61M 25/0097; A61M 39/288
USPC 604/43

(57) ABSTRACT

A catheter hub includes a hub body defining a first lumen and a second lumen. At least a portion of the first lumen defines a first longitudinal axis, and at least a portion of the second lumen defines a second longitudinal axis. A cap coupled to the hub body is configured and arranged in a non-intersecting orientation relative to the first and second longitudinal axes. A first arm and a second arm each includes a distal portion coupled to the hub body, and a proximal portion which is movable, in a direction toward a respective longitudinal axis, into releasable engagement with the cap.

### TECHNICAL FIELD

The present disclosure relates to catheters, and more particularly, to controlling flow through catheters.

### BACKGROUND

Catheters can be used for the introduction and withdrawal of fluids to and from body cavities, ducts and vessels. For example, hemodialysis catheters can be used to withdraw blood from a blood vessel for dialysis treatment and to return the treated blood to a blood vessel for recirculation. Clamps for hemodialysis catheters are placed about extension tubes in fluid communication with one or more lumens of the catheter. During catheter placement and periods between use (interdialytic periods), the clamps are clamped about the extension tubes to retain a flush solution (e.g., heparinized saline) within the catheter and to seal the catheter lumen(s).

It can be difficult for a clinician to locate and operate the catheter clamps to clamp the respective extension tubes, particularly when the clinician has only one hand available. This can impact the time and complexity associated with a medical procedure.

### SUMMARY

A hemodialysis catheter hub includes integrated clamping structure which replaces extension tube clamps of a catheter assembly.

In one aspect, a catheter hub comprises a hub body defining a first lumen and a second lumen. At least a portion of the first lumen defines a first longitudinal axis, and at least a portion of the second lumen defines a second longitudinal axis. A cap is coupled to the hub body and is configured and arranged in a non-intersecting orientation relative to the first and second longitudinal axes. A first arm and a second arm each include a distal portion coupled to the hub body and a proximal portion which is movable in a direction toward a respective longitudinal axis into releasable engagement with the cap.

In some embodiments, the cap is coupled to the hub body in the area defined between the first and second longitudinal axes.

In certain embodiments, the first and second lumens each have a respective proximal portion closest to the cap and a respective distal portion farthest from the cap, and at least the proximal portions of the first and second lumens define an oblique angle therebetween.

In some embodiments, the distal portions of the first and second lumens are substantially parallel to one another. In certain embodiments, the proximal portions of the first and second lumens define the respective first and second longitudinal axes.

In some embodiments, the proximal portion of each of the first and second lumens has a circular cross-section, and the distal portion of each of the first and second lumens has a semi-circular cross-section.

In certain embodiments, at least a portion of the first lumen is defined by a first hypo tube at least partially disposed in the hub body, and at least a portion of the second lumen is defined by a second hypo tube at least partially disposed in the hub body.

In some embodiments, the hub body defines a suture wing channel.

In certain embodiments, the hub body tapers in a direction from the proximal portion of the hub body to a distal portion of the hub body.

In some embodiments, the cap comprises a first rack portion defining a first orifice axially aligned with the first longitudinal axis, a second rack portion defining a second orifice axially aligned with the second longitudinal axis, wherein the first and second arms are movable toward one another into releasable engagement with the respective first and second rack portions.

In certain embodiments, each rack portion comprises at least one tooth engageable with a respective one of the first and second arms to resist movement of the respective arm in a direction away from a respective one of the longitudinal axes.

In some embodiments, each rack portion is movable in a proximal direction to release engagement of the respective first and second arms.

In certain embodiments, the cap is coupled to the body between the first rack portion and the second rack portion.

In some embodiments, the cap defines a first recessed area and a second recessed area and, the first and second arms are movable into releasable engagement with the respective first and second recessed areas.

In certain embodiments, the first and second recessed areas are disposed in the area defined between the first and second longitudinal axes.

In some embodiments, a sheath disposed over the cap and body.

In another aspect, a catheter assembly includes a catheter hub having a hub body defining at least two lumens wherein at least a portion of each lumen defines a respective longitudinal axis. A cap coupled to the hub body and is configured and arranged in a non-intersecting orientation relative to the at least two longitudinal axes. The catheter assembly includes at least two arms, each arm corresponding to a respective lumen. A distal portion of each arm is coupled to the hub body and a proximal portion of each arm has a free end. Additionally, the catheter assembly includes at least two extension tubes, each extension tube is in fluid communication with a respective lumen and defines a center axis parallel to a respective longitudinal axis. The proximal portion of each arm is movable in a direction toward a respective longitudinal axis into releasable engagement with the cap in a position occluding the respective extension tube.

In certain embodiments, the at least two lumens each have a distal portion substantially parallel to one another and a proximal portion defining the respective longitudinal axis.

In some embodiments, the proximal portion of each lumen has a circular cross-section, and the distal portion of each lumen has a semi-circular cross-section.

In certain embodiments, at least a portion of each lumen is defined by a hypo tube at least partially disposed in the hub body.

Embodiments can include one or more of the following advantages.

In some embodiments, the clamps are integrated into the hub body. This will remove the need for extension tube clamps and make it easier for a clinician to locate and operate both clamps simultaneously using a single hand.

In certain embodiments, the hub body is encapsulated in a sheath. This sheath seals the hub body from unwanted outside influence while maintaining the dexterous functioning of the hub body.

Other features, objects, and advantages will be apparent from the description and drawings, and from the claims.

### BRIEF DESCRIPTION OF THE DRAWINGS

FIG. 1 is a schematic representation of a catheter assembly shown placed within a patient during a medical procedure.

FIG. 2 is a side isometric view of the catheter assembly of FIG. 1, with a clamp in an unengaged position.

FIG. 3 is a side, cross-sectional view of a catheter hub body of the catheter assembly of FIG. 1.

FIG. 4A is a partial side cross-sectional view of the catheter assembly of FIG. 1, with a distal end portion of an elongated catheter body cutaway and the clamp structure in an unengaged position.

FIG. 4B is an isometric view of a hypo tube of the catheter assembly of FIG. 1.

FIG. 4C is a cross-sectional view of the hypo tube of FIG. 4B, taken along section line 4C-4C of FIG. 4B.

FIG. 4D is a cross-sectional view of the hypo tube of FIG. 4B, taken along section line 4D-4D of FIG. 4B.

FIG. 4E is a cross-sectional view of the catheter of FIG. 4A, taken along section line 4E-4E of FIG. 4A.

FIG. 5 is a side cross-sectional view of the catheter assembly of FIG. 1, with the clamp in a releasably engaged position.

FIG. 6 is a side isometric view of a catheter assembly with a clamp in an unengaged position.

FIG. 7 is a side isometric view of the catheter assembly of FIG. 6, with a cap disengaged from a hub body.

FIG. 8 is a side cross-sectional view of the cap and hub body of the catheter assembly of FIG. 6, with the cap and hub body assembled and the clamp in an unengaged position.

FIG. 9 is side cross-sectional view of the catheter assembly of FIG. 6, with a distal portion of the elongated catheter body cut away and the clamp in an unengaged position.

FIG. 10 is a side view of the catheter assembly of FIG. 6 with the distal portion of the elongated catheter body cut away and the clamp in an engaged position.

FIG. 11 is a side cross-sectional view of the catheter assembly of FIG. 6, with the clamp in an engaged position.

FIG. 12 is a side isometric view of a catheter assembly including a sheath.

Like reference symbols in the various drawings indicate like elements.

### DETAILED DESCRIPTION

In the discussion that follows, the term “proximal” refers to a portion of a structure that is closer to a practitioner while the structure is in use. The term “distal” refers to a portion of the structure that is further from the practitioner while the structure is in use. The term “subject” refers to a human patient or other animal, and the term “clinician” refers to a doctor, nurse or other care provider and may include support personnel.

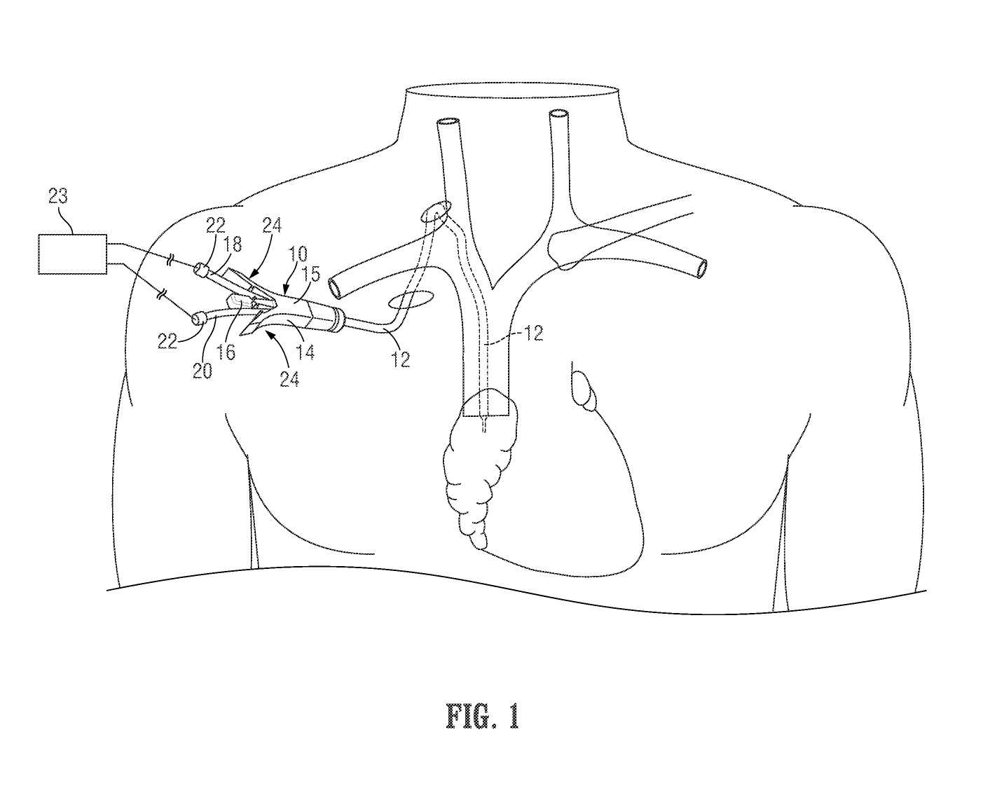

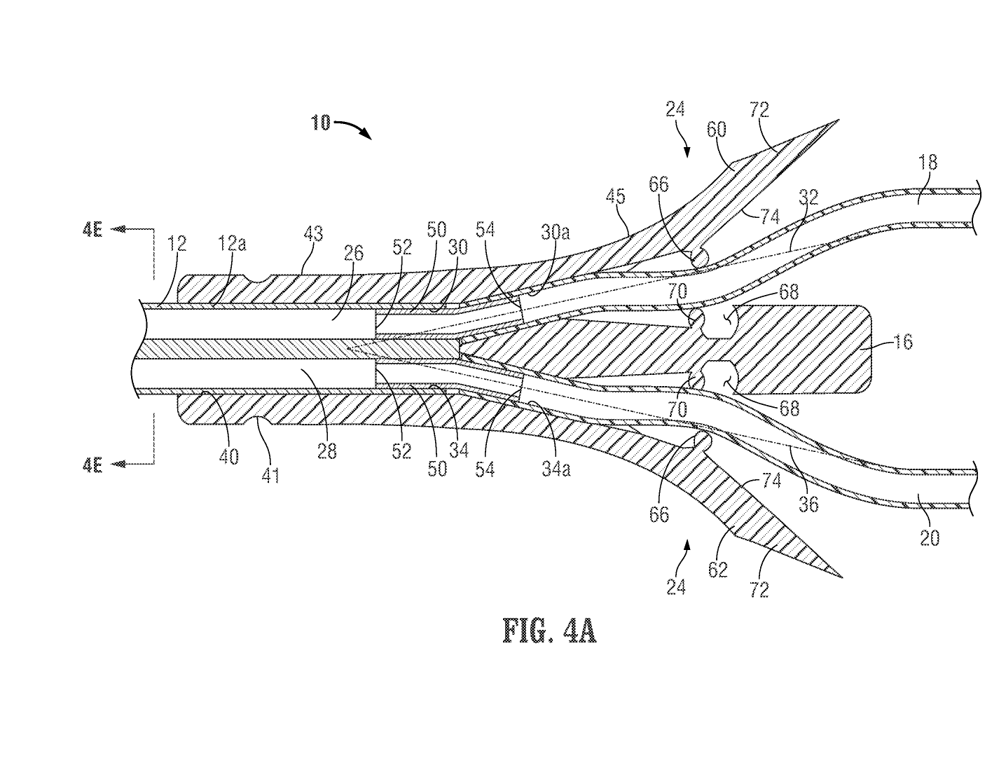

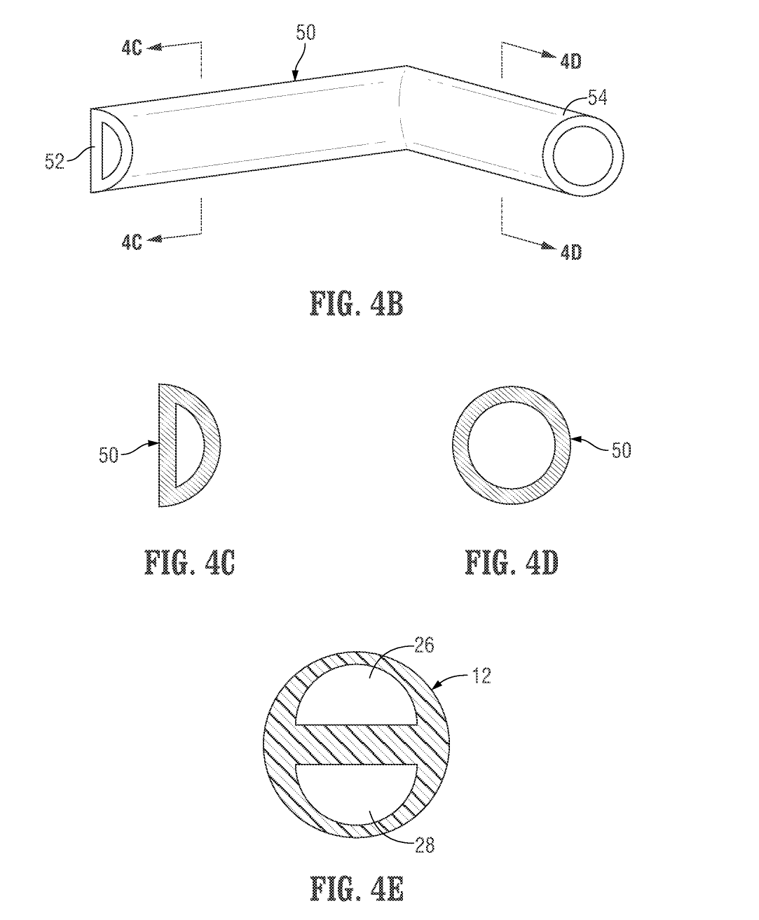

Referring to FIGS. 1-5, a catheter assembly 10 includes an elongated catheter body 12, a catheter hub 14 including a hub body 15, a cap 16, and first and second extension tubes 18, 20. The cap 16 is coupled to the hub body 14, and each of the extension tubes 18, 20 includes a connector 22, which can connect the extension tubes 18, 20 to a medical device 23. The connector 22 can be, for example, a luer connector and the medical device 23 can be a dialyzer. The catheter hub 14 includes a clamp 24 operable to clamp each of the extension tubes 18, 20. The extension tubes 18, 20 are in fluid communication with respective catheter lumens 26, 28 (FIG. 4) defined by elongated catheter body 12 such that operation of the clamp 24 controls the flow of fluid through the lumens 26, 28.

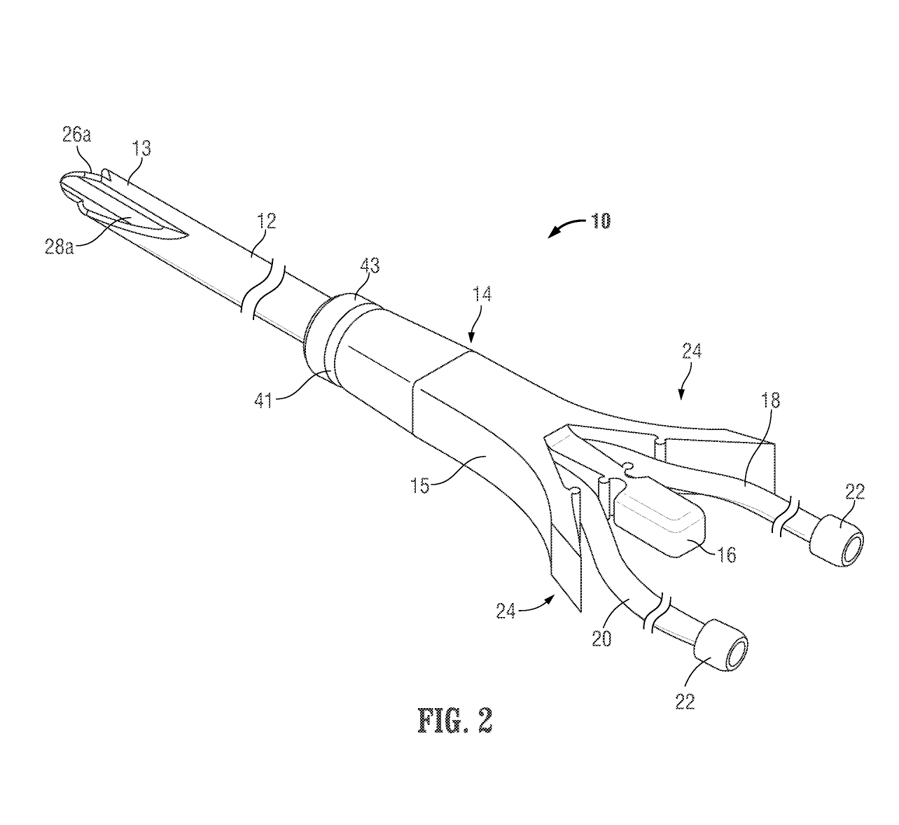

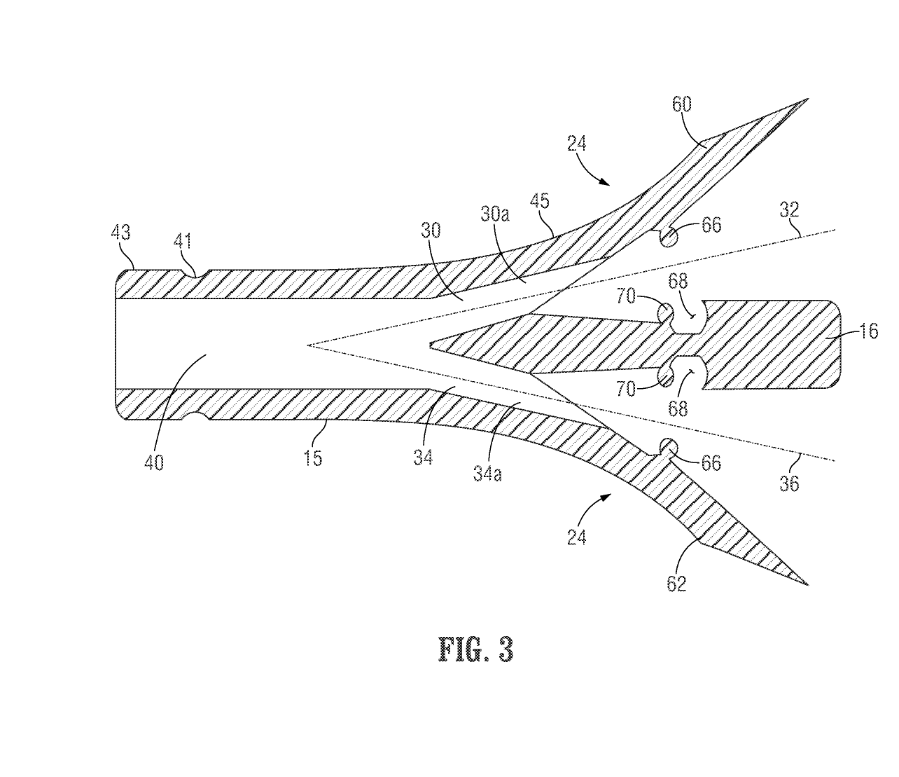

Referring now to FIGS. 2 and 3, the hub body 15 defines a first lumen 30 having a proximal portion 30a adjacent the cap 16 and a second lumen 34 having a proximal portion 34a adjacent the cap 16. The proximal portion 30a of the first lumen 30 defines a first longitudinal axis 32, and the proximal portion 34a of the second lumen 34 defines a second longitudinal axis 36. The cap 16 is in non-intersecting relation with the first and second longitudinal axes 32 and 36. As used herein, the non-intersecting relation between the cap 16 and the first and second longitudinal axes 32 and 36 includes the first and second longitudinal axes 32 and 36 extending proximally past and in spaced relation from the cap 16. The first and second longitudinal axes 32 and 36 define an oblique angle β (e.g., an acute angle). The first and second lumens 30, 34 have a common distal portion 40 distal to the cap 16.

A suture wing channel 41 is defined at the distal end of the hub body 15. The suture wing channel 41 provides a recess into which a suture (not shown) is receivable to secure the distal end portion of the hub body 15 to a patient after placement of the catheter assembly 10. The suture wing channel 41 provides strain relief at the distal end portion of the hub body 15. For example, as compared to a catheter assembly without a suture wing channel, the suture wing channel 41 can reduce the movement of the implanted portion of the catheter assembly 10 as a clinician exerts force on the clamp 24 and hub body 15 to move the clamp 24 between open and closed positions.

Referring to FIGS. 2, 3 and 4A-4E, the common distal portion 40 of the hub body 15 is dimensioned to receive a proximal end portion 12a of the elongated catheter body 12 of the catheter assembly 10. The elongated catheter body 12 defines two lumens 26 and 28, each terminating with a respective opening 26a and 28a at the distal end portion 13 of the elongate catheter body 12. Each of the lumens 26 and 28 of the elongate catheter body 12 has a semi-circular cross-section along its length while the elongate catheter body 12 has a substantially circular cross-section along its length. Each of the proximal portions 30a and 34a of the first and second lumens 30, 34, respectively, of the hub body 15 receives a distal end portion of a respective extension tube 18, 20, each of which has a circular cross-section.

A respective hypo tube 50 extends from within each of the proximal portions 30a and 34a of the first and second lumens 30 and 34 into the common distal portion 40 of the hub body 15. Each hypo tube 50 has a distal end portion 52 having a semi-circular cross-section received in the proximal end portion 12a of the elongated catheter body 12 and a proximal end portion 54 having a circular cross-section received within respective distal end portions of the extension tubes 18, 20. The distal end portions 52 of the respective hypo tubes 50 extend parallel to each other, and the proximal end portions 54 of the hypo tubes 50 extend parallel to the respective longitudinal axis 32, 36 of the respective lumen 30, 34 in which the hypo tube 50 is disposed. The hypo tube 50 fluidly couples the extension tubes 18, 20 to the respective lumens 28 and 26 of the elongated catheter body 12 in a fluid tight manner.

The catheter hub 14 can be molded about the proximal end portion 12a of the elongated catheter body 12, the hypo tubes 50, and the distal end portions of the extension tubes 18, 20 to fixedly secure the components of the catheter assembly 10 together. The hub body 15 tapers in a direction from the proximal portion to the distal portion of the hub body 15. This taper can facilitate single-handed application of force to the clamp 24 by a clinician operating the catheter assembly 10. For example, the clinician can support a palm on the distal portion of the hub body to counteract the force of the clinician's fingers acting on the clamp 24.

The clamp 24 is integrated onto the catheter hub 14 and includes a first arm 60 and a second arm 62. A distal portion of each of the first and second arms 60, 62 is coupled to a proximal portion of the hub body 15 in cantilevered fashion such that a proximal portion of each arm 60, 62 is movable toward a respective longitudinal axis 32, 36 of the first and second lumens 30, 34 of the hub body 15 and toward the cap 16 of the catheter hub 14. The unitary construction of the first and second arms 60, 62 to the hub body 15 can facilitate single-handed transmission of force to the first and second arms 60, 62. Additionally or alternatively, as compared to catheter assemblies that make use of separate clamps, the unitary construction of the first and second arms 60, 62 to the hub body 15 can reduce the profile of the catheter assembly 10, which can improve patient comfort during interdialytic periods.

Each of the first and second arms 60, 62 includes a projection 66 which extends from an inner surface of each arm 60, 62 towards the cap 16. The clamp 24 also includes first and second recessed areas 68 defined within the cap 16. Each of the recessed areas 68 is configured for releasable engagement with a respective projection 66 when the arms 60, 62 are moved towards the cap 16 and across the longitudinal axis 32, 36 of the first or second lumen 30, 34. A resilient engagement member 70 is supported on the cap 16 adjacent each recessed area 68. Each engagement member 70 is flexible to allow the extension tube 18, 20 and a respective projection 66 to enter a respective recessed area 68. The engagement member 70 abuts the respective extension tube 18, 20 when the respective extension tube 18, 20 is pressed into releasable engagement within the respective recessed area 68 to secure the extension tubes 18, 20 in an occluded position.

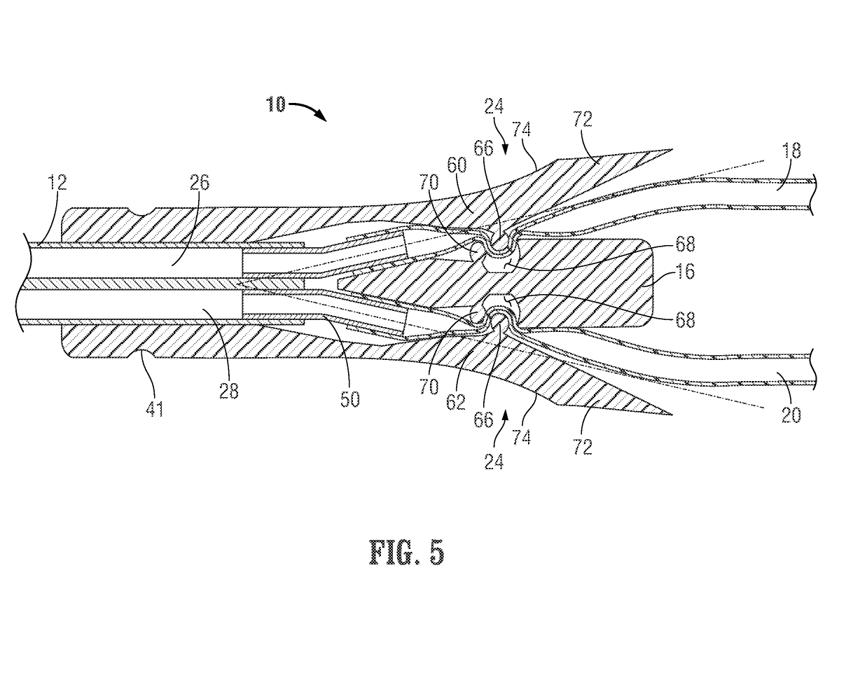

Referring to FIG. 5, in use, a syringe can be used to introduce a flush solution (e.g., heparin) through the extension tubes 18, 20 and into the catheter lumens 26, 28. The clamp 24 can be operated, as described in further detail below, to clamp the extension tubes 18, 20 such that the flush solution remains in the catheter assembly 10 during catheter placement. After the elongated catheter body 12 is placed within the patient, the connectors 22 can connect the respective extension tubes 18, 20 to the medical device 23 and the clamp 24 can be disengaged to unclamp the extension tubes 18, 20 such that the medical device 23 can provide medical treatment (e.g., dialysis treatment) to a patient.

After the medical treatment, the clamp 24 can be operated to occlude the extension tubes 18, 20 and the extension tubes 18, 20 can be disengaged from the medical device 23. With the extension tubes 18, 20 disconnected from the medical device 23, a syringe can be connected to the extension tubes 18, 20 and the clamp 24 disengaged from the extension tubes 18, 20 to introduce flush solution to the catheter lumens 26, 28. The clamp 24 can be operated again to seal the extension tubes 18, 20 during an interdialytic period.

As compared to clamps that are not integrated with a catheter hub, the integrated clamps 24 of the catheter hub 14 can facilitate single hand operation by a clinician to occlude and/or release both extension tubes 18, 20 simultaneously using a single hand. Such single hand operation can, for example, simplify medical procedures. For example, the clinician can grasp the arms 60, 62 between the thumb and fingers or between fingers and a palm of a single hand and compress the arms 60, 62 toward the cap 16. When this occurs, the projection 66 on each arm 60, 62 engages an extension tube 18, 20 and presses the extension tube 18, 20 into releasable engagement with a recessed area 68 on the cap 16. As the extension tube 18, 20 is pressed into the recessed area 68, the resilient engagement member 70 can be deflected distally to allow the extension tube 18, 20 and projection 66 to enter the recess 68 such that the extension tube 18, 20 is compressed between the projection 66 and the recessed area 68 to occlude the extension tubes 18, 20. The resilient nature of the engagement member 70 and/or the projection 66 retains each clamp 24 in an engaged state to retain the extension tubes 18, 20 in an occluded condition.

To release the protrusions 66 from the recessed areas 68 and, thus, unclamp the extension tubes, proximal end portions 72 of the arms 60, 62 can be pressed (e.g., between a clinician's thumb and fingers) towards the cap 16. When this occurs, respective central portions 74 of arms 60, 62 will move outwardly from cap 16 as the respective proximal end portions 72 of arms 60, 62 engage the cap 16 to release the respective extension tubes 18, 20 and protrusions 66 from the recessed areas 68.

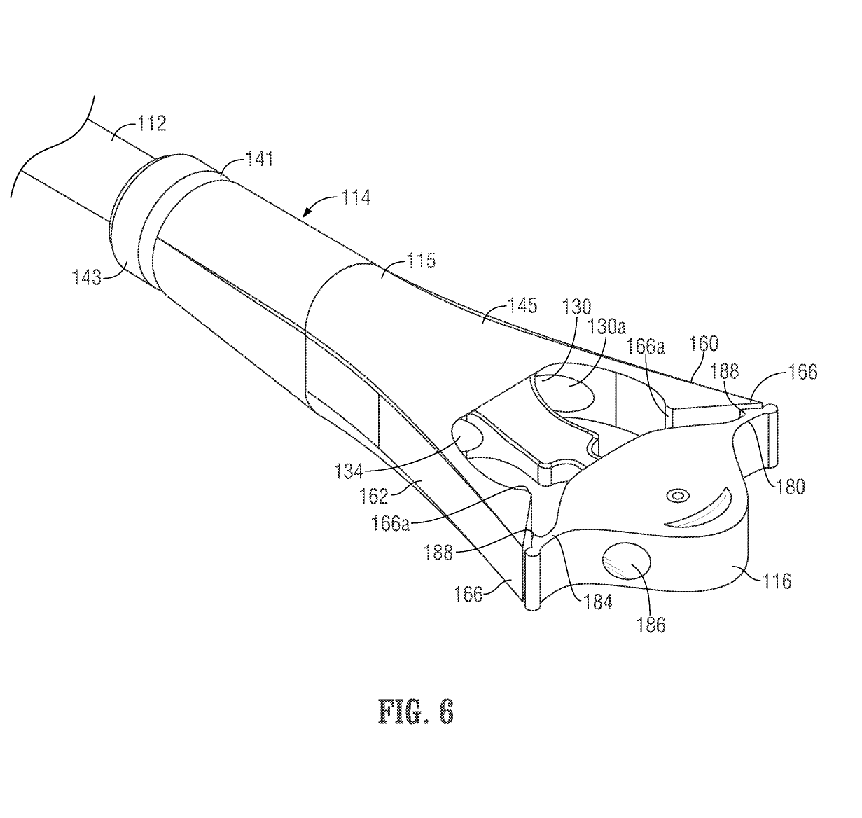

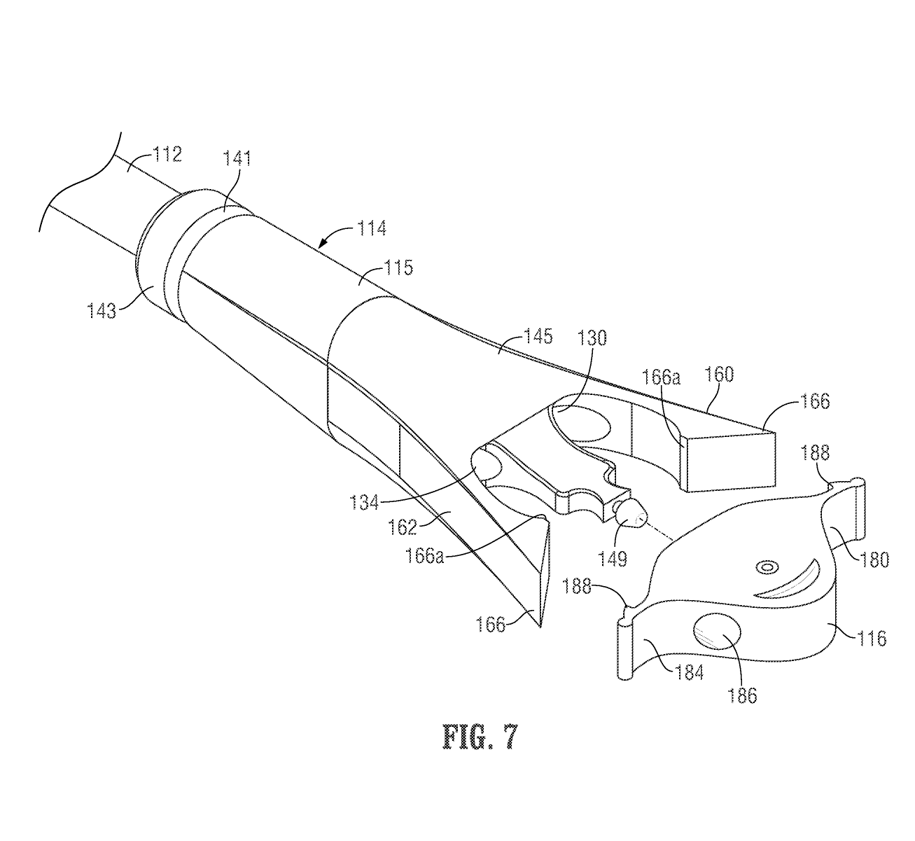

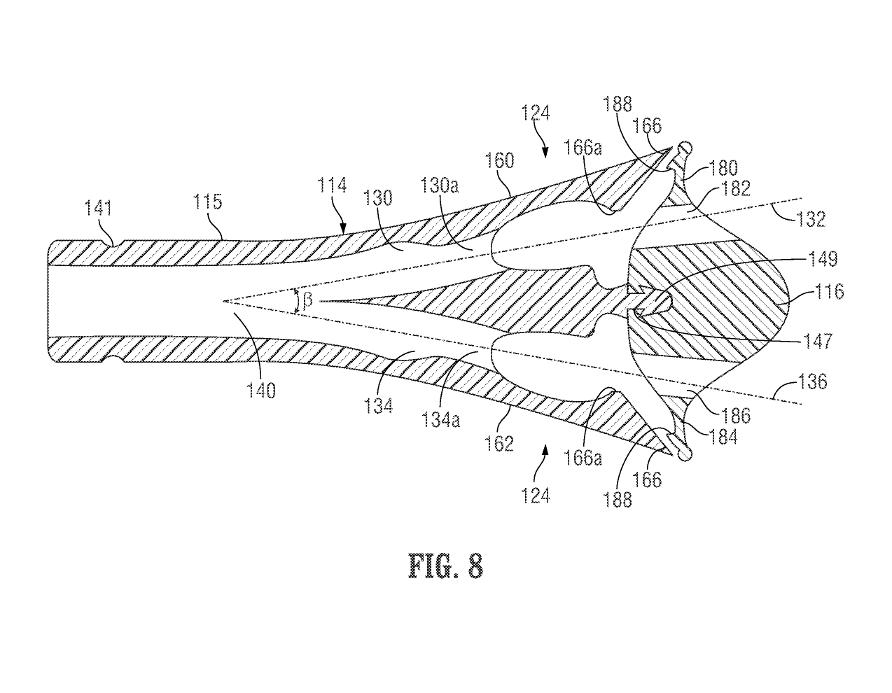

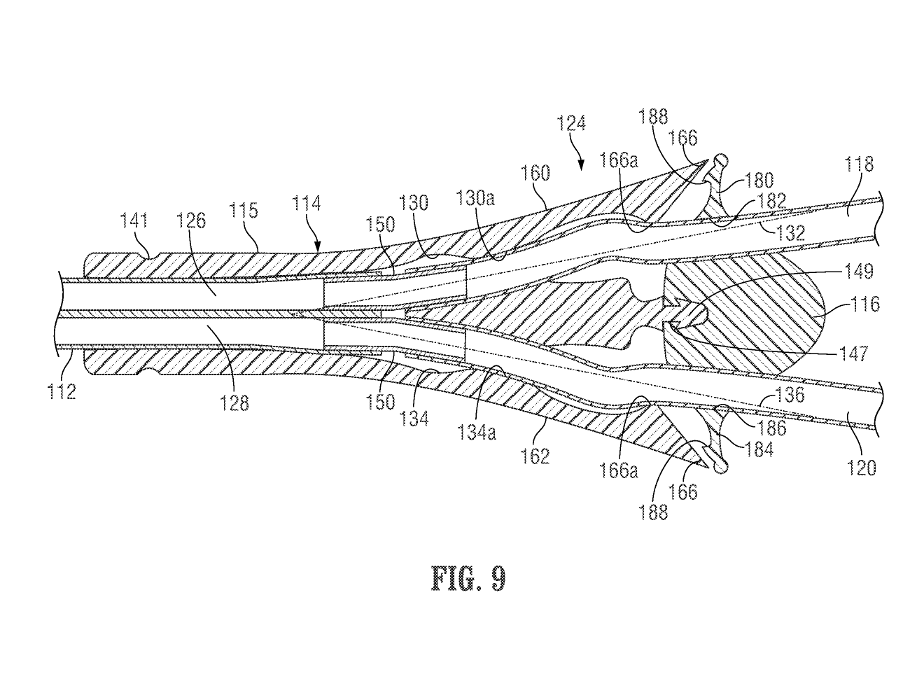

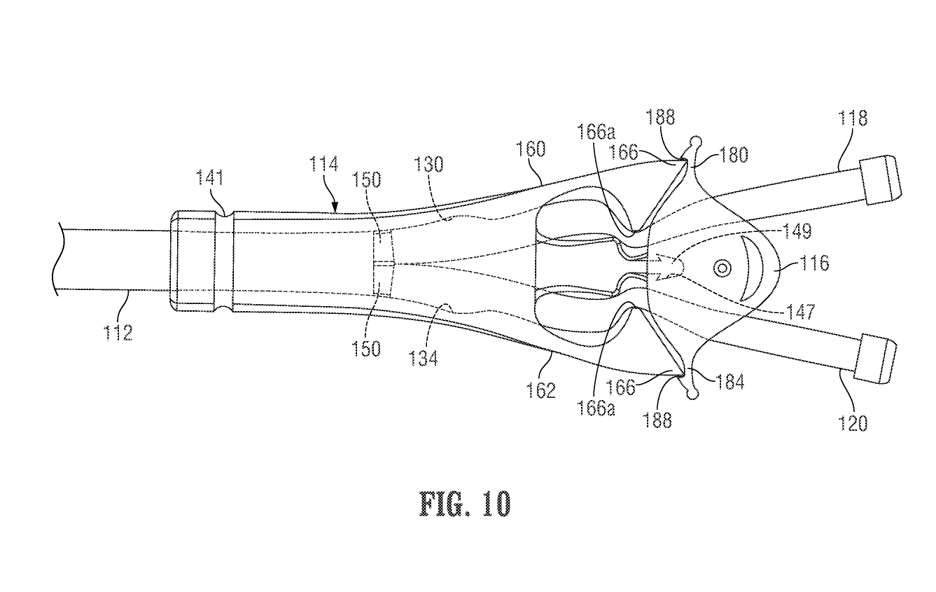

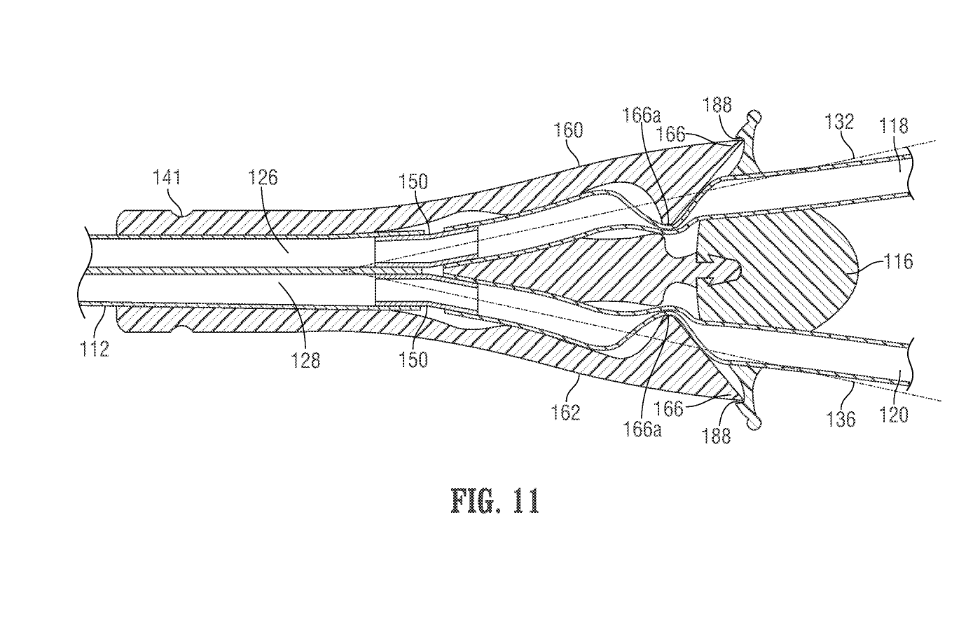

While certain embodiments have been described, other embodiments are possible. For example, while catheter assemblies have been described as including a clamp defining first and second recessed areas, other configurations are additionally or alternatively possible. For example, referring to FIGS. 6-11, a catheter assembly 110 includes an elongated catheter body 112, a catheter hub 114, and first and second extension tubes 118, 120. The catheter hub 114 includes a hub body 115 and a cap 116 coupled to the hub body 115. The elongated catheter body 112 defines first and second lumens 126 and 128. The elongated catheter body 112 and extension tubes 118, 120 are analogous to the elongated catheter body 12 and extension tubes 18, 20 described above with respect to catheter assembly 10 (FIGS. 1-5).

The hub body 115 defines a first lumen 130 and a second lumen 134. The first lumen has a proximal portion 130a disposed toward the cap 116 and defining a first longitudinal axis 132. The second lumen 134 has a proximal portion 134a disposed toward the cap 116 and defining a second longitudinal axis 136. The first and second longitudinal axes 132 and 136 are in non-intersecting relation with the cap 116 and together define an oblique angle β (e.g., an acute angle). The first and second lumens 130, 134 each terminate at a common distal portion 140 spaced distally from the cap 116.

The hub body 115 and the cap 116 can be formed (e.g., molded) separately and secured together mechanically. For example, the cap 116 can define a stepped recess 147 into which a stepped plug 149, formed on the hub body 115, is receivable to fixedly secure the cap 116 to the hub body 115.

A suture wing channel 141 is defined at the distal end portion 143 of the hub body 115. The suture wing channel 141 provides a recess for receiving a suture (not shown) to secure the distal end of the hub body 115 to a patient after placement of the catheter assembly 110. The suture wing channel 141 can provide strain relief at the distal end portion 143 of the hub body 115 in a manner analogous to the strain relief provided by the suture wing channel 41 (FIGS. 1-5).

The clamp 124 is integrated onto the catheter hub 114 and includes a first arm 160 and a second arm 162. A distal portion of the first and second arms 160, 162 is coupled to a proximal portion 145 of the hub body 115 in cantilevered fashion such that a proximal portion 166 of each arm 160, 162 is movable toward a respective longitudinal axis 132, 136 of the first and second lumens 130, 134 of the hub body 115 and toward the cap 116 of the catheter hub 114.

The cap 116 includes a first rack portion 180 and a second rack portion 184. The first rack portion 180 defines a first orifice 182 axially aligned with the first longitudinal axis 132, and the second rack portion 184 defines a second orifice 186 axially aligned with the second longitudinal axis 136. The first and second arms 160 and 162 are movable towards one another into releasable engagement with the first and second rack portions 180, 184. For example, the first and second arms 160, 162 are movable toward the cap 160 such that the respective proximal edge 166 of each of the arms 160, 162 engages at least one tooth 188 of each of the first and second rack portions 180, 184 to deflect the rack portions 180, 184 proximally. Proximal movement of the first and second rack portions 180, 184 allows the respective proximal edge 166 of each arm 160, 162 to pass over and move into locking engagement with the at least one tooth 188 of the respective first and second rack portions 180, 184.

Each of the extension tubes 118, 120 includes a distal end portion disposed within the catheter hub 114 and fluidly coupled to the proximal end portion of the elongated catheter body 112 by a respective hypo tube 150. The hypo tubes 150 are analogous to hypo tubes 50 (FIGS. 1-5) described above. Each extension tube 118, 120 extends proximally from the respective hypo tube 150, through the respective lumens 130, 134, and through the respective orifices 182 and 186 of the cap 116.

Each arm 160, 162 includes an engagement surface 166a. When the arms 160, 162 are compressed together, the engagement surface 166a of each arm 160, 162 engages and compresses a respective extension tube 118, 120 against the cap 116 to occlude the extension tube 118, 120.

As another example, while one tooth 188 is shown as extending from each of the first and second rack portions 180, 184, other configurations are additionally or alternatively possible. For example, a plurality of teeth can extend from one or both of the first and second rack portions to facilitate selectively controlling the degree of occlusion provided in the extension tube or tubes.

As yet another example, while caps and hub bodies 115 have been described as being separate components, other configurations are additionally or alternatively possible. For example, a cap and a hub body can be integrally formed to form a catheter hub. Additionally or alternatively, the hub body can be molded about a proximal end portion of an elongated catheter body, distal end portions of extension tubes and/or hypo tubes.

As yet another example, while the catheter assemblies have been described as including respective hypo tubes, other configurations are additionally or alternatively possible. For example, a catheter assembly can be formed without hypo tubes.

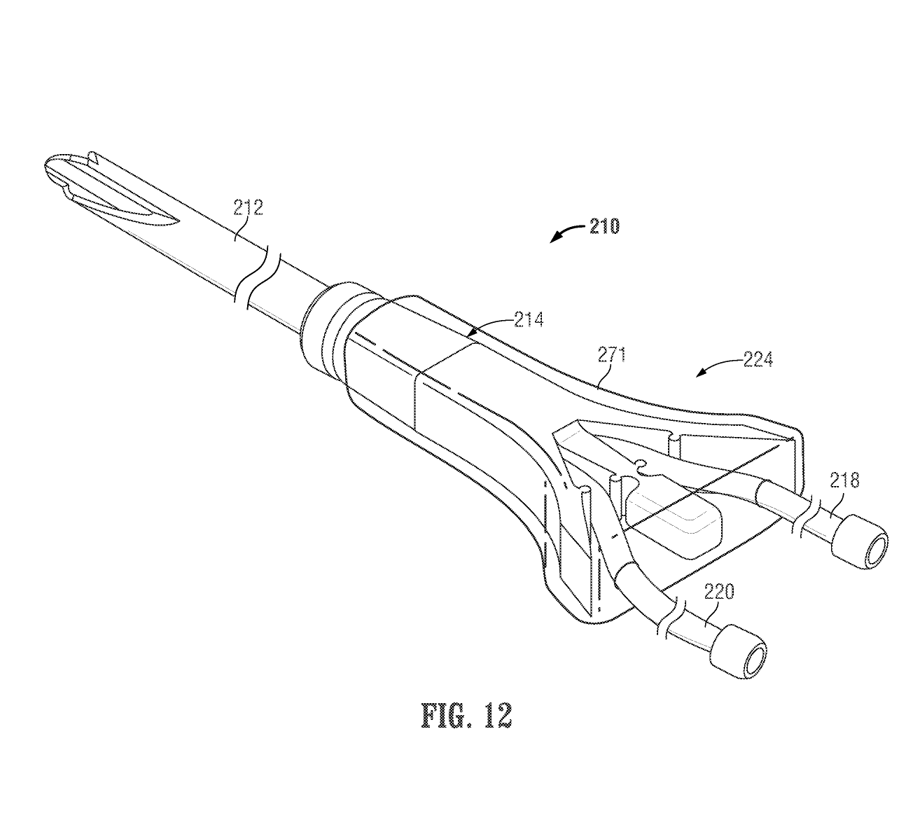

As another example, while catheter hubs have been described as being exposed, other arrangements are additionally or alternatively possible. For example, referring to FIG. 12, a catheter assembly 210 can include a sheath 271 disposed over a catheter hub 214, including a clamp 224, distal end portions of extension tubes 218, 220, and/or proximal end portions of an elongated catheter body 212. In use, the sheath 271 can seal off the catheter hub 214 from unwanted debris and/or infectious material. The sheath 271 is compressible to facilitate operation of the clamp 224 through the sheath 271. The clamp 224 is analogous to clamp 24 (FIGS. 1-5).

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A catheter hub comprising:
a hub body defining a first lumen and a second lumen, at least a portion of the first lumen defining a first longitudinal axis, and at least a portion of the second lumen defining a second longitudinal axis;
a cap coupled to the hub body, the cap configured and arranged in a non-intersecting orientation relative to the first and second longitudinal axes; and
a first arm and a second arm, a distal portion of each arm coupled to the hub body, and a proximal portion of each arm movable, in a direction toward a respective longitudinal axis, into releasable engagement with the cap.

2. The catheter hub of claim 1, wherein the cap is coupled to the hub body in the area defined between the first and second longitudinal axes.

3. The catheter hub of claim 1, wherein the first and second lumens each have a respective proximal portion closest to the cap and a respective distal portion farthest from the cap, and at least the proximal portions of the first and second lumens define an oblique angle therebetween.

4. The catheter hub of claim 3, wherein the distal portions of the first and second lumens are substantially parallel to one another.

5. The catheter hub of claim 3, wherein the proximal portions of the first and second lumens define the respective first and second longitudinal axes.

6. The catheter hub of claim 3, wherein the proximal portion of each of the first and second lumens has a circular cross-section, and the distal portion of each of the first and second lumens has a semi-circular cross-section.

7. The catheter hub of claim 1, wherein at least a portion of the first lumen is defined by a first hypo tube at least partially disposed in the hub body, and at least a portion of the second lumen is defined by a second hypo tube at least partially disposed in the hub body.

8. The catheter hub of claim 1, wherein the hub body defines a suture wing channel.

9. The catheter hub of claim 1, wherein the hub body tapers in a direction from the proximal portion of the hub body to a distal portion of the hub body.

10. The catheter hub of claim 1, wherein the cap comprises a first rack portion defining a first orifice axially aligned with the first longitudinal axis, a second rack portion defining a second orifice axially aligned with the second longitudinal axis, and the first and second arms are movable toward one another into releasable engagement with the respective first and second rack portions.

11. The catheter hub of claim 10, wherein each rack portion comprises at least one tooth engageable with a respective one of the first and second arms to resist movement of the respective arm in a direction away from a respective one of the longitudinal axes.

12. The catheter hub of claim 10, wherein each rack portion is movable in a proximal direction to release engagement of the respective first and second arms.

13. The catheter hub of claim 10, wherein the cap is coupled to the body between the first rack portion and the second rack portion.

14. The catheter hub of claim 1, wherein the cap defines a first recessed area and a second recessed area, the first and second arms movable into releasable engagement with the respective first and second recessed areas.

15. The catheter hub of claim 14, wherein the first and second recessed areas are disposed in the area defined between the first and second longitudinal axes.

16. The catheter hub of claim 1, further comprising a sheath disposed over the cap and body.

17. A catheter assembly comprising:
a catheter hub including
a hub body defining at least two lumens, at least a portion of each lumen defining a respective longitudinal axis,
a cap coupled to the hub body, the cap configured and arranged in a non-intersecting orientation relative to the at least two longitudinal axes, and
at least two arms, each arm corresponding to a respective lumen, a distal portion of each arm coupled to the hub body and a proximal portion of each arm having a free end; and
at least two extension tubes, each extension tube in fluid communication with a respective lumen and defining a center axis parallel to a respective longitudinal axis, the proximal portion of each arm movable in a direction toward a respective longitudinal axis into releasable engagement with the cap in a position occluding the respective extension tube.

18. The catheter assembly of claim 17, wherein the at least two lumens each have a distal portion substantially parallel to one another and a proximal portion defining the respective longitudinal axis.

19. The catheter assembly of claim 18, wherein the proximal portion of each lumen has a circular cross-section, and the distal portion of each lumen has a semi-circular cross-section.

20. The catheter assembly of claim 17, wherein at least a portion of each lumen is defined by a hypo tube at least partially disposed in the hub body.
